(19) United States
(12) Plant Patent Application Publication
(10) Pub. No.: US 2012/0284886 P1
(43) Pub. Date: Nov. 8, 2012

(54) Azalea plant named 'Stewla#1'

(50) ;

(76) Inventor: Larry Steward, Wooster, OH (US)

(21) Appl. No.: 13/068,201

(22) Filed: May 5, 2011

(51) Int. Cl. A01H 5/00

(52) U.S. Cl. PLT/238

(57) ABSTRACT

A new and distinct cultivar of Azalea plant named ‘Stewla#1’, characterized by its globe to broad columnar plant form; freely branching habit; terminal truss flower arrangement; pure white hose-in-hose flowers with some yellow-green flecking in the throat of the petals; green-yellow flower buds; and hardiness in USDA zone 6a.

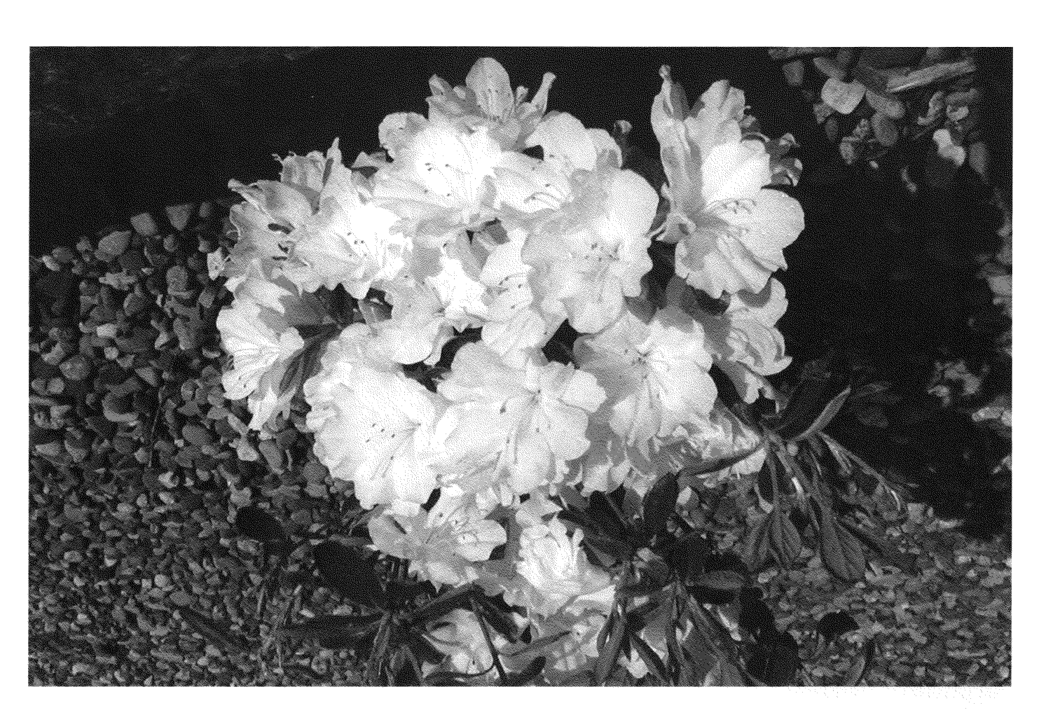

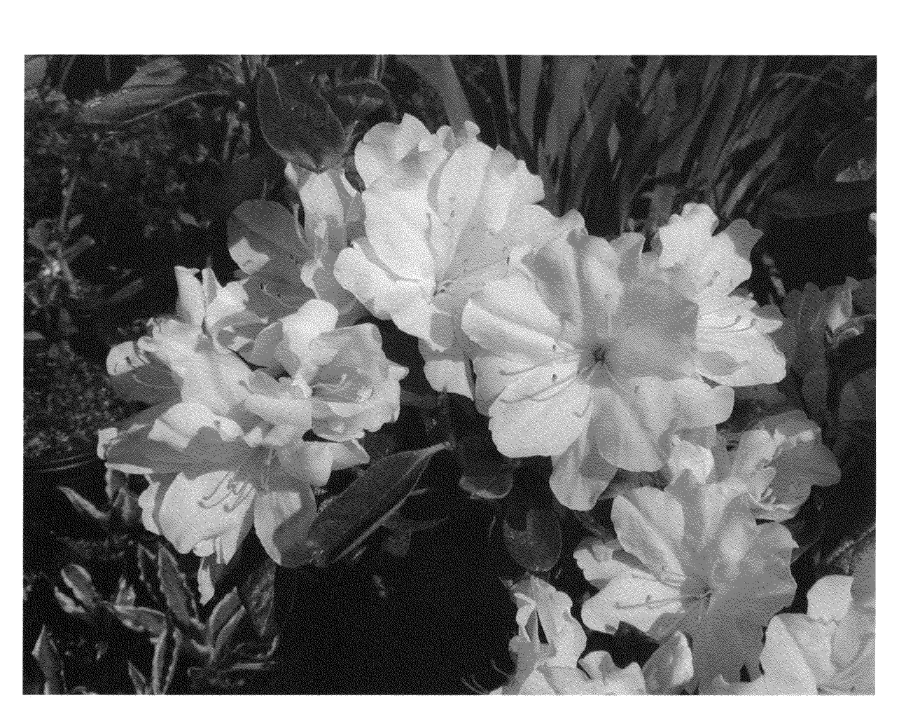

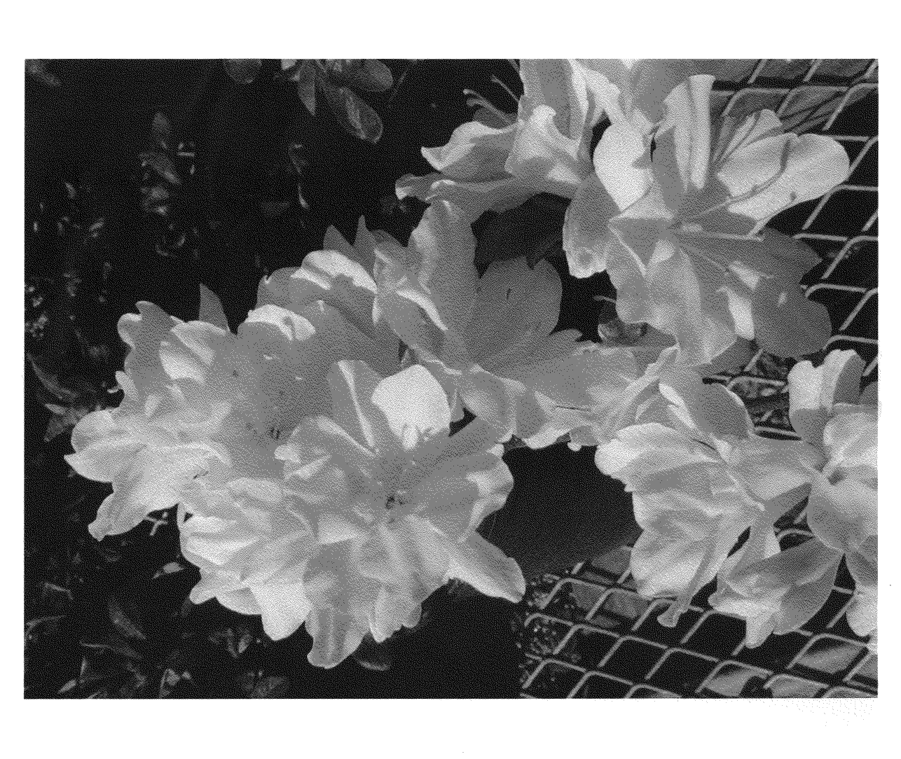

### CROSS REFERENCE TO RELATED APPLICATIONS

This application makes no claim of priority to any earlier filed application.

### STATEMENT REGARDING FEDERALLY-SPONSORED RESEARCH AND DEVELOPMENT

Not applicable.

### LATIN NAME OF THE GENUS AND SPECIES OF THE PLANT CLAIMED

Rhododendron obtusum.

### CULTIVAR DENOMINATION

Stewla#1.

### BACKGROUND OF THE INVENTION

The present invention relates to a new and distinct cultivar of Azalea, botanically known as Rhododendron obtusum, hereinafter referred to as ‘Stewla#1.’ Stewla#1 is an evergreen greenhouse-forcing type Azalea and may be used for outside landscape planting.

The new Azalea was discovered by the inventor in Charlottesville, Va. in a controlled, landscape planting of Delaware Valley White Azaleas in 1974. The exact parentage of Stewla#1 is unknown. Compared to plants of the white, single-flowered commercial cultivar ‘Delaware Valley White,’ the new Azalea has white hose-in-hose flowers borne in trusses with some yellow-green flecking in the throat of the petals and green-yellow emerging buds.

Asexual reproduction of the new Azalea by hardwood, semi-hardwood and softwood vegetative cuttings taken in a controlled environment in Charlottesville, Va. since 1975 and in Wooster, Ohio since 1998 have shown that the unique features of this new Azalea are stable and reproduced true to type in successive generations.

### SUMMARY OF THE INVENTION

The following traits have been repeatedly observed and are determined to be the most outstanding and distinguishing characteristics of ‘Stewla#1.’ These traits distinguish ‘Stewla#1’ as a new and distinct cultivar:

1. Globe to broad columnar plant form.

2. Freely branching habit.

3. Terminal truss flower arrangement.

4. Pure white hose-in-hose flowers with some yellow-green flecking in the throat of the petals.

5. Green-yellow flower buds.

6. Subtle fruity scent.

7. Hardy in USDA zone 6a.

### BRIEF DESCRIPTION OF THE DRAWINGS

The new Azalea is illustrated by the accompanying photographic prints in which:

1. The photograph on the first sheet displays a view of a typical flowering plant of ‘Stewla#1.’

2. The photograph on the second sheet displays a close-up view of typical flowers of “Stewla#1.”

3. The photograph on the third sheet displays a close-up view of typical flowers of ‘Stewla#1’ and includes a view of a typical green-yellow flower bud in the lower righthand area of the photograph.

Colors in the photographs may differ slightly from the color values cited in the detailed botanical description, which accurately describes the colors of the new Azalea.

### DETAILED BOTANICAL DESCRIPTION

The following observations, measurements, values, and comparisons are based on observations made of a plant found in an established landscape planting in Charlottesville, Va. between 1975 and 2010 and plants asexually reproduced in commercial production practices at Wooster, Ohio between 1998 and 2010. The plant found in an established landscape planting in Charlottesville, Va. was exposed to temperatures ranging from 39.4° C. in 1977 to −23.3° C. in 1994.

In the following description, color references are made to The Royal Horticultural Society Colour Chart, 1995 Edition, except where general terms of ordinary dictionary significance are used.
Botanical classification: Rhododendron obtusum ‘Stewla#1’.
Commercial classification: Evergreen ornamental flowering medium shrub for outside landscape planting or for greenhouse-forcing pot azalea.
Parentage:
   Female or seed parent.—Unknown.
   Male or pollen parent.—Unknown.
Propagation:
   Type.—Hardwood, semi-hardwood and softwood vegetative cuttings.
   Time to initiate roots.—About 4 to 6 weeks.
   Time to develop roots.—About 6 to 10 weeks.
   Root description.—Fine, fibrous white.
   Rooting habit.—Freely spreading and dividing.
Plant description:
   Plant form and growth habit.—Globe to broad columnar.
   Branching habit.—Freely branching.
   Plant height, soil level to top of flowers.—About 198 cm.
   Plant diameter, area of spread.—About 194 cm.
   Lateral branch description.—Length: About 91 cm. Diameter at base: About 5 mm. Internode length. — About 19.1 mm. Strength: Strong. Texture: Young: medium. Texture: Mature: fine. Color: Young: Close to Green Group 143C. Color: Mature: Close to Green Group 141A.
   Foliage description.—Arrangement: Alternate. Foliage retention: Very good. Length: About 5.72 cm. Width: About 2.22 cm. Shape: oval. Apex: mucronate. Base: acuminate. Margin: Entire to slightly toothed. Venation pattern: branched/net. Texture: pubescent underside, very glossy upper side upon maturity on the upper side of the leaves. Color: Young foliage, upper surface: Close to Green Group 143C. Young foliage, lower surface: Close to Green Group 143C. Mature foliage, upper surface: Close to Green Group 141A. Mature foliage, lower surface: Close to Green Group 141C. Petiole: Length: 0″ as leaf is sessile.
Flower description:
   Natural flowering season.—April in Hardiness Zone 5.
   Flower arrangement.—In a terminal truss defined as a domed cluster of flowers or flower mass.
   Flower fragrance.—Very slight but sweet.
   Flower appearance.—Hose-in-hose flower form with a joined whorl of petals in a second whorl of petaloid sepals (transformed sepals), pure white flower with some yellow-green flecking in the throat of the petals.
   Flower diameter.—About 3.81 to 5.08 cm
   Flower depth.—About 3.81 cm.
   Postproduction longevity.—About 5 to 7 days.
   Flower bud (before petal color is showing).—Buds point outward from a central attachment, are greenish-yellow, and are quite showy even before flowers open.
   Flower bud (just starting to show petal color).—Rate of opening: About 2 days. Length: About 2.54 cm. Diameter: About 9.5 mm. Shape: Oval. Color: Close to Green-Yellow group 1-B.
   Petals.—Arrangement: Five petals joined in a whorl within the whorl of petaloid sepals. Length: About 2.54 cm. Width: About 12.7 mm. Shape: oval. Margin: entire. Texture: glabrous. Color, when opening, upper surface: Close to Green-Yellow Group 154-B to White. Color, when opening, lower surface: White with throat of close to Green-Yellow Group 1-C. Color, fully opened, upper surface: Clear White. Color, fully opened, lower surface: Clear White with slight blotch of Green-Yellow Group 1-C.
   Petaloids.—Quantity: None.
   Sepals.—Petal like. Arrangement: Five joined in a whorl. Shape: oval. Margin: Entire. Texture: Glabrous. Color, upper surface: Pure White. Color, lower surface: Pure White.
   Peduncles.—Length: About 6.35 mm. Diameter: About 1.59 mm. Angle: About 90 degrees. Strength: Strong. Texture: Glabrous. Color: Green.
   Reproductive organs.—Androecium: Quantity of stamens per flower: Five. Anther size: About 0.79 mm. Anther shape: Oval. Anther color: White. Filament length: About 9.53 mm. Filament color: White. Pollen amount: Unknown. Pollen color: Close to Greyed-Orange Group 163-D. Gynoecium: Pistil quantity: One. Pistil length: Close to 3.18 mm. Stigma shape: Oval. Stigma color: Close to Yellow Group 13-C. Style length: About 3.18 mm. Style color: Green. Ovary color: Green.
   Seed.—Very fine brown.
Disease/pest resistance: Resistant to most common diseases found on outside azaleas. Slightly susceptible to azalea lace bug.
Drought resistance: Very resistant to drought.
Weather/temperature tolerance: Tolerated temperatures from −23° C. to 39° C. in outside environment.

It is claimed:

1. A new and distinct Azalea plant named ‘Stewla#1’ as illustrated and described.
